(19) United States
(12) Patent Application Publication
(10) Pub. No.: US 2023/0329601 A1
(43) Pub. Date: Oct. 19, 2023

(54) CATHETER INCLUDING A SENSOR CONFIGURED TO SENSE A BIOMARKER

(71) Applicant: Covidien LP, Mansfield, MA (US)

(72) Inventors: David J. Miller, Austin, TX (US); Søren Aasmul, Holte (DK); Jesper Svenning Kristensen, Virum (DK)

(21) Appl. No.: 18/040,104

(22) PCT Filed: Sep. 1, 2021
(86) PCT No.: PCT/US2021/048686

§ 371 Date: Jan. 31, 2023

Related U.S. Application Data
(60) Provisional application No. 63/074,755, filed on Sep. 4, 2020.

(51) Int. Cl. A61B 5/1486; A61B 5/145

(52) U.S. Cl. CPC A61B 5/14865; A61B 5/14546; A61M 25/04

(57) ABSTRACT

In some examples, a system comprises a catheter including an elongated body defining a lumen, the elongated body comprising a proximal portion and a distal portion; an anchoring member positioned on the proximal portion of the elongated body, the anchoring member configured to anchor the proximal portion of the elongated body to a patient; and a sensor configured to sense a biomarker in a fluid within the lumen of the elongated body, wherein the biomarker is indicative of an acute kidney infection (AKI) of the patient.

This application claims priority from U.S. Provisional Patent Application No. 63/074,755, filed Sep. 4, 2020, the entire content of which is incorporated herein by reference.

### TECHNICAL FIELD

This disclosure relates to medical devices, more particularly, to catheters.

### BACKGROUND

Medical devices, such as catheters, may be used to assist a patient in voiding their bladder. In some instances, such catheters may be used before, during, and/or after surgery. In the case of using a catheter to assist a patient in voiding their bladder, a Foley catheter is a type of catheter used for longer time periods than a non-Foley catheter. Some Foley catheters are constructed of silicon rubber and include an anchoring member, that may be an inflatable balloon inflated in a patient's bladder to serve as an anchor, so a proximal end of the catheter does not slip out of the patient's bladder.

The disclosure describes catheters (e.g., a Foley catheter), systems including catheters, and techniques for making and using such catheters and catheter systems. A catheter system may include a sensor configured to sense a biomarker (e.g., a biomarker concentration) in a fluid within a lumen of the catheter. The biomarker may be indicative of an acute kidney infection (AKI) of the patient. The sensor may allow for in situ sensing (e.g., on substantially continuous or periodic basis) of the AKI biomarker.

In one example, the disclosure relates to a catheter comprising an elongated body defining a lumen, the elongated body comprising a proximal portion and a distal portion; an anchoring member positioned on the proximal portion of the elongated body, the anchoring member configured to anchor the proximal portion of the elongated body to a patient; and a sensor configured to sense a biomarker in a fluid within the lumen of the elongated body, wherein the biomarker is indicative of an acute kidney infection (AKI) of the patient.

In another example, the disclosure relates to a method comprising sensing, via a sensor, a biomarker in a fluid within a lumen of a catheter, the catheter comprising: an elongated body defining the lumen, the elongated body comprising a proximal portion and a distal portion; an anchoring member positioned on the proximal portion of the elongated body, the anchoring member configured to anchor the proximal portion of the elongated body to a patient; and the sensor configured to sense the biomarker in a fluid within the lumen of the elongated body, wherein the biomarker is indicative of an acute kidney infection (AKI) of the patient.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example medical device system including an example catheter in accordance with an example of the disclosure.

FIG. 2 is a conceptual diagram illustrating example a cross-section of the medical device of FIG. 1, the cross-section being take along line A-A of FIG. 1.

FIG. 3 is conceptual diagram illustrating a portion of an example catheter including a sampling lumen in accordance with an example of the disclosure.

FIG. 4 is a conceptual diagram illustrating a competitive assay process that may be used to sense an AKI biomarker according to an example of the disclosure.

FIG. 5 is a conceptual diagram illustrating an example AKI biomarker sensor using a surface bound sensing principle.

FIG. 6 is a conceptual diagram illustrating an example AKI biomarker sensor using a homogenous assay (not surface bound).

FIG. 7 is a conceptual schematic diagram illustrating an example competitive binding assay in accordance with examples of the disclosure.

FIG. 8 is a conceptual schematic diagram illustrating how the equilibrium state of an example competitive binding assay may be determined in accordance with examples of the disclosure.

FIG. 9 is a conceptual schematic diagram illustrating an example frequency domain lifetime interrogation technique in accordance with examples of the disclosure.

FIG. 10 is a conceptual schematic diagram illustrating an example intensity interrogation technique in accordance with examples of the disclosure.

FIG. 11A is a conceptual schematic diagram illustrating example intensity interrogation instrumentation in accordance with some examples of the disclosure.

FIG. 11B is a diagram illustrating example fluorescence emission sensed by the intensity interrogation instrumentation of FIG. 11A.

### DETAILED DESCRIPTION

In general, the disclosure describes medical devices and systems including a catheter, such as a Foley catheter or other urinary or non-urinary catheter, and methods of using the same. As will be described below, examples of the disclosure may include catheters having one or more sensors configured to sense, e.g., a concentration of one or more biomarkers within a fluid that is within a lumen of the catheter, such as a drainage lumen. For ease of description, examples of the disclosure are primarily described with regard to a catheter such as a Foley catheter being employed as a urinary catheter within a patient. The examples, however, can be applicable to other types and uses of catheters and sensing one or more biomarkers present in a fluid that is within a lumen of the respective catheters.

Acute kidney injury (AKI) is a complication that can occur after major surgeries such as cardiac surgery and other operations that are long and involve significant blood loss or fluid shifts. One cause of surgery-associated AKI may be hypoxia of the kidneys. Renal hypoxia may cause degradation of renal function, that, e.g., after one to three days, may cause a reduced urine output and/or an accumulation of waste products in the bloodstream. This accumulation of fluid and waste products may delay the recovery of the patient leading to more extended and expensive hospital stays and sometimes requiring renal replacement therapy.

One approach to preventing AKI is to monitor the oxygenation status of a patient's kidneys. However, accurate monitoring may be challenging due to the inaccessibility of the kidneys that are deep in the abdominal cavity. Near-Infrared spectroscopy (NIRS) may measure regional oximetry and may have some utility in infants and slender adults but does not have the depth of penetration and specificity required for most adults.

Systemic vital signs like cardiac output, blood pressure, and hematocrit may be useful but may not always be sufficient to properly monitor the kidneys. When the body becomes stressed, such as during cardiac surgery, blood flow may be reduced to vital organs in a reliable sequence based on the criticality of the organs. It has been observed that the skin may be the first to realize reduced blood flow, followed by the intestines and then the kidneys, then the brain and then the heart. The skin and the intestines may withstand short hypoxic episodes and recover normal function, but the kidneys may be damaged with even brief hypoxic episodes.

In some examples, the amount of one or more biomarkers in the urine of a patient, such as, neutrophil gelatinase-associated lipocalin (NGAL), tissue inhibitor of metalloproteinase 2 (TIMP-2), or insulin-like growth factor binding protein 7 (IGFMP7) may be determined to monitor the patient for AKI. For example, the relative concentration of such biomarkers in patient's urine may indicate the patient is experiencing or will likely be experiencing AKI. The level of an AKI biomarker in a patient's urine may be estimated by collecting a urine sample from a patient, transporting the sample to a laboratory, testing the urine sample in a laboratory setting, and then reporting the results in a manner that a clinician can review. However, such a process may take a relatively long time and only provide an estimate of the level of AKI biomarker in the patient's urine at the point in time the sample was collected. To monitor the level of the AKI biomarker over a period of time using such a process may require taking frequent urine samples from the patient, transporting those samples to the laboratory setting for testing, and then subsequently reporting the results of each sample to a clinician, as described above.

Examples of the present disclosure are related to device features that aid in the monitoring AKI biomarkers in the urine (or other fluid) of a patient using a catheter device such as a urinary catheter. In some examples, the catheter includes a sensor that is configured to sense an amount of a biomarker (e.g., concentration or change in concentration of the biomarker over time). The biomarker sensor may be incorporated into the catheter in a manner that allows for in situ testing of a patient's urine rather than requiring the urine be sample and transported to a laboratory. In some examples, the catheter device may be configured to sense the level of biomarker in a patient's urine on a substantially continuous basis. In some examples, the catheter device may be configured to determine an amount of a biomarker in the urine of a patient using such a sensor in an on-demand basis, e.g., in response to instructions inputted a clinician or other user. The results from the sensing operation may be outputted and displayed to a user, e.g., via a user interface of an external device communicatively coupled to the catheter device.

In some examples, a catheter may including multiple sensors and/or a single sensor that senses more than one biomarker. In one example, a catheter employ a combination of sensors utilizing several competitive assays; homogeneous or surface bound, and a creatinine biosensor which can provide monitoring of the AKI biomarkers directly using the catheter in a continuous fashion, in contrast to the lab tests which may be spot checks only.

In some examples, the sensor may be configured to directly analyze a patient's urine to determine the level of biomarker within the urine without the urine being removed from the catheter system. For example, the sensor may be configured such that a surface of the sensor interacts with urine within a drainage lumen of an elongated body of the catheter so that the sensor may determine the level of a biomarker in the patient's urine while it flows from the patient's bladder to an external drainage collection bag via the catheter drainage lumen.

In some examples, the catheter includes a sampling lumen as a side lumen or bypass lumen that is fluidically connected to a primary drainage lumen of the catheter. A portion of urine flowing through the drainage lumen may be introduced into the sampling lumen so that the portion of the urine interacts with a biomarker sensor within the sampling lumen flow path. In some examples, the sampling lumen may be configured such that the sampled urine is returned to the drainage lumen, e.g., so that the sampling lumen urine ultimately flows into a collection bag fluidically coupled to the drainage lumen. In such an example, the sampled urine may bypass a portion of the drainage lumen so sampling but still end up in the collection bag coupled to the drainage lumen by either flowing back into the drainage lumen or via a more direct connection to the collection bag. In other examples, the sampling lumen may be coupled to another collection bag that may be separately emptied or replaced once filled.

In some examples, the catheter device may include one or more valves that may be actuated to control flow into the sampling lumen. For example, the catheter device may include a valve at an inlet to the sampling lumen and/or a valve at the outlet of the sampling lumen. In some examples, the valves may be selectively actuated (e.g., under the control of a user such as a clinician) so that urine flows into the sampling lumen for a period of time during which the sensor determines the level of biomarker within the urine. The valves may be closed such that urine from the drainage lumen does not flow into the sampling lumen when the sensor is not determining the level of biomarker in the patient's urine. In some examples, the valves may be automatically controlled (e.g., opened and closed) by control circuitry electrically connected to the valves. The control circuitry can be part of an existing patient monitoring device or can be part of a separate monitoring device. In other examples, the valves may be manually controlled by a clinician or other user.

Any suitable sensing configured to monitor an AKI biomarker may be employed in an example catheter device. In some examples, the sensor includes a competitive assay using a surface-bound analyte binder and a soluble ligand. The ligand binds to the surface in competition with the presence of the AKI biomarkers. The amount of ligand binding can be sensed and by doing so the amount of biomarker can be measured. In some examples, a homogenous assay (not surface bound) may be used.

Example of the present disclosure may provide one or more advantages. For example, the advantage of monitoring the AKI biomarkers with a catheter device (e.g., in an in situ basis) is that the AKI biomarker measurement may be continuous when the catheter is inserted within the patient, in contrast to the lab tests which are spot checks only as described above. Example catheter devices of the present disclosure may also generate a sensor reading quickly, conveniently, and one that is presented directly to the clinician or other user, e.g., at a bedside of the patient, at a central station, or the like.

As noted above, a Foley catheter may be a type of urinary catheter used in the examples of the present disclosure. A Foley catheter may be modified in the manner described herein to allow for sensing of AKI biomarkers, e.g., on an in situ basis. In some examples, one or more AKI biomarker sensors may be used in conjunction with a Foley Catheter to monitor renal function. In some examples, the sensor(s) may provide data indicating allowing relatively early detection of acute kidney injury in a patient, e.g., to allow for treatment of the AKI injury in a timely manner.

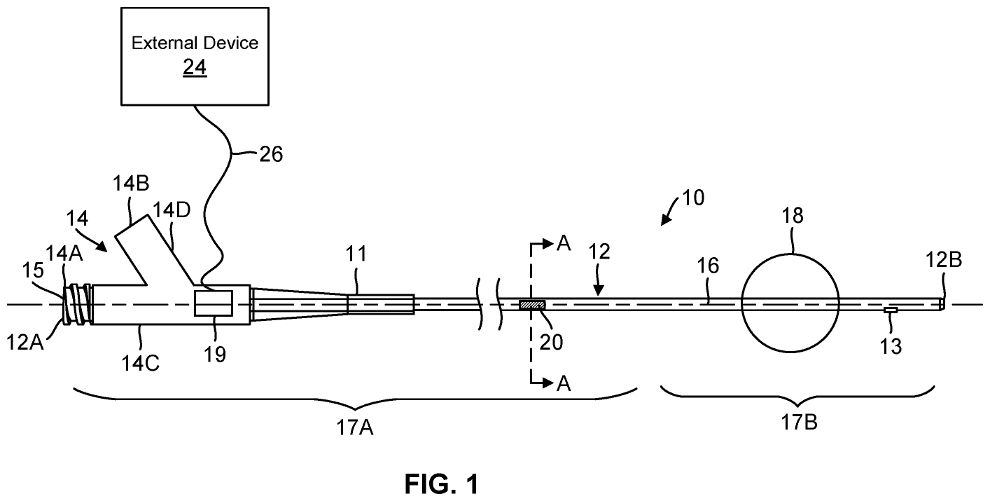

FIG. 1 is a conceptual side elevation view of an example medical device 10, that includes elongated body 12 including hub 14, and anchoring member 18. In some examples, medical device 10 is a catheter, such as a Foley catheter. Medical device 10 may also be referred to herein as catheter 10 or catheter device 10. While a Foley catheter and its intended use is primarily referred to herein to describe medical device 10, in other examples, medical device 10 may be used for other purposes, such as to drain wounds or for intravascular monitoring or medical procedures.

Elongated body 12 includes a distal portion 17A and a proximal portion 17B. Distal portion 17A includes a distal end 12A of elongated body 12 and is intended to be external to a patient's body when in use, while proximal portion 17B includes a proximal end 12B of elongated body 12 and is intended to be internal to a patient's body when in use. For example, when proximal portion 17B is positioned within a patient, e.g., so proximal end 12B of elongated body 12 is within the patient's urethra and bladder, distal portion 17A may remain outside of the body of the patient.

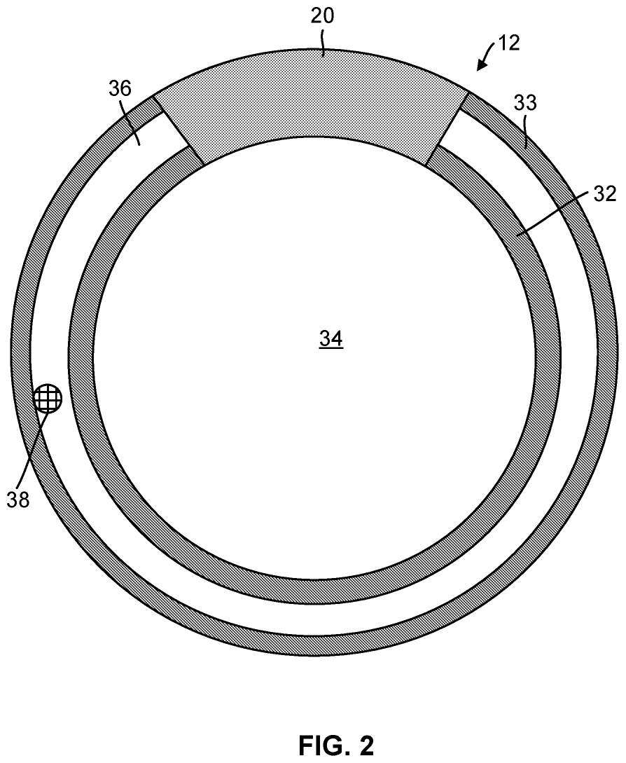

As shown in FIG. 1, elongated body 12 may be a body extending from distal end 12A to proximal end 12B and that defines one or more inner lumens. In the example shown in FIGS. 1 and 2, elongated body 12 defines lumen 34 and lumen 36 (shown in FIG. 2). In some examples, lumen 34 may be a drainage lumen configured to drain a fluid from a target site, such as to drain urine from a bladder. In other examples lumen 34 may be used for any other suitable purpose, such as to deliver a substance or another medical device to a target site within a patient. Lumen 34 may extend from fluid opening 13 to fluid opening 14A. Both fluid opening 13 and fluid opening 14A may be fluidically coupled to lumen 34, so a fluid may flow from one of fluid opening 13 or fluid opening 14A to the other of fluid opening 13 or fluid opening 14A through lumen 34. In the example where lumen 34 is a drainage lumen, fluid opening 13 and fluid opening 14A may be drainage openings.

In some examples, elongated body 12 has a suitable length for accessing the bladder of a patient through the urethra. The length may be measured along central longitudinal axis 16 of elongated body 12. In some examples, elongated body 12 may have an outer diameter of about 12 French to about 14 French, but other dimensions may be used in other examples. Distal and proximal portions 17A, 17B of elongated body 12 may each have any suitable length.

Elongated body 12 may be structurally configured to be relatively flexible, pushable, and relatively kink- and buckle-resistant, so it may resist buckling when a pushing force is applied to a relatively distal portion of medical device 10 to advance elongated body 12 proximally through the urethra and into the bladder. Kinking and/or buckling of elongated body 12 may hinder a clinician's efforts to push the elongated body proximally. Any suitable material may be used for elongated body 12, such as a suitable biocompatible polymer or other biocompatible material.

In the example shown in FIG. 1, distal end 12A of elongated body 12 includes hub 14 and is mechanically connected to hub 14 via an adhesive, welding, or another suitable technique or combination of techniques. Hub 14 defines an opening through which the one or more inner lumens (e.g., lumen 34 shown in FIG. 2) of elongated body 12 may be accessed and, in some examples, closed. While hub 14 is shown in FIG. 1 as having two arms, 14C and 14D (e.g., a “Y-hub”), hub 14 may have any suitable number of arms, that may depend on the number of inner lumens defined by elongated body 12. For example, each arm may be fluidically coupled to a respective inner lumen of elongated body 12. In the example of FIG. 1, hub 14 comprises a fluid opening 14A, that is fluidically coupled to lumen 34, and an inflation opening 14B, that is fluidically coupled to an inflation lumen 36 (shown in FIG. 2) of elongated body 12. In examples in which anchoring member 18 does not include an expandable balloon, rather than defining inflation lumen 36, elongated body 12 may define an inner lumen configured to receive a deployment mechanism (e.g., a pull wire or a push wire) for deploying an expandable structure anchoring member 18 and hub 14 may comprise fluid opening 14A and an opening 14B via which a clinician may access the deployment mechanism.

In examples in which medical device 10 is a Foley catheter, a fluid collection container (e.g., a urine bag) may be attached directly or indirectly to fluid opening 14A for collecting urine draining from the patient's bladder. Inflation opening 14B may be operable to connect to an inflation device to inflate anchoring member 18 positioned on proximal portion 17B of medical device 10. Anchoring member 18 may be uninflated or undeployed when not in use. Hub 14 may include connectors, such as connector 15, for connecting to other devices, such as the fluid collection container and the inflation source. In some examples, medical device 10 includes strain relief member 11, that may be a part of hub 14 or may be separate from hub 14.

Proximal portion 17B of medical device 10 comprises anchoring member 18 and fluid opening 13. Anchoring member 18 may include any suitable structure configured to expand from a relatively low profile state to an expanded state in that anchoring member 18 may engage with tissue of a patient (e.g., inside a bladder) to help secure and prevent movement of proximal portion 17B out of the body of the patient. For example, anchoring member 18 may include an anchor balloon or other expandable structure. When inflated or deployed, anchoring member 18 may function to anchor medical device 10 to the patient, for example, within the patient's bladder. In this manner, the portion of medical device 10 on the proximal side of anchoring member 18 may not slip out of the patient's bladder. Fluid opening 13 may be positioned on the surface of longitudinal axis of medical device 10 between anchoring member 18 and the proximal end 12B (as shown) or may be positioned at the proximal end 12B.

As described herein, medical device 10 includes sensor 20 configured to sense (e.g., determine) an amount (e.g., concentration or activity) of an AKI biomarker within a fluid such as urine flowing within lumen 34 of elongate body 16. In some examples, catheter 10 includes memory 19 on hub 14 or another portion of catheter 10, or memory 19 may be physically separate from catheter 10 but electrically connected thereto. Memory 19 may store all or a portion of information related to the AKI biomarker(s) sensed by sensor 20. Sensor information stored on memory 19 may be communicated with external device 24. In some examples, memory 19 may also store program instructions, such as software or algorithms, that may include one or more program modules, that are executable by processing circuitry (not shown in FIG. 1). When executed by the processing circuitry, such program instructions may cause the processing circuitry and external device 24 to provide the functionality ascribed to them herein. The program instructions may be embodied in software and/or firmware. Memory 19 may include any volatile, non-volatile, magnetic, optical, or electrical media, such as a random access memory (RAM), read-only memory (ROM), non-volatile RAM (NVRAM), electrically erasable programmable ROM (EEPROM), flash memory, or any other digital media.

In some examples, catheter 10 may have processing circuitry on elongated body 12 including hub 14 that is configured to control all or some operations of sensor 20. In some examples, the processing circuitry of external device 24 may control all or some operations of sensor 20. In some examples, the processing circuitry of external device 24 and processing circuitry of catheter 10 may control all or some of operations of sensor 20 together. Connection 26 (e.g., a wired or a wireless connection) may communicate information between memory 19 and external device 24.

The processing circuitry of catheter device 10 and/or external device 24 may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, the processing circuitry may include multiple components, such as any combination of one or more microprocessors, one or more DSPs, one or more ASICs, or one or more FPGAs, as well as other discrete or integrated logic circuitry, and/or analog circuitry.

In accordance with examples of the disclosure, catheter 10 includes one or more sensors 20 configured to monitor the level (e.g., concentration or activity) of AKI biomarkers(s) of a fluid within lumen 34 (FIG. 2) of elongate body 12. The AKI biomarker may be a substance that may be present (or present above a certain level) when AKI is occurring or is likely to occur in one or more kidneys of a patient. Example AKI biomarkers may include neutrophil gelatinase-associated lipocalin (NGAL), tissue inhibitor of metalloproteinase 2 (TIMP-2), or insulin-like growth factor binding protein 7 (IGFMP7). In some examples, creatinine, creatine, or urea may be AKI biomarkers. In some examples, sensor 20 may include a competitive assay or other sensing component that allows sensor 20 to determine an amount or presence of AKI biomarker within a fluid within lumen 34 of catheter 10. For example, urine within lumen 34 may come into contact with sensor 20, and sensor 20 may determine an amount (e.g., concentration or activity) of one or more AKI biomarkers within the urine.

In some examples, sensor 20 may be representative of a single sensor or multiple sensors. Where sensor 20 may be multiple sensors, the multiple sensors may be located on elongated body 12 of catheter 10 at the same location or at different locations despite being shown at a single location in FIG. 1. Sensor 20 may communicate information indicative of a sensed parameter (e.g., a biomarker) to processing circuitry of catheter 10 and/or external device 24, and/or may communicate the information to memory 19. For example, sensor 20 may communicate sensor information to memory 19 and memory 19 to external device 24 via an electrical, optical, wireless, or other connection 26. Connection 26 may be an electrical, optical, wireless, or other connection. In some examples, sensor 20 may communicate sensor information to memory 19 through connection(s) within elongated body 12 of medical device 10 from proximal portion 17B to distal portion 17A via embedded wire(s) or optical cable(s). In other examples, sensor 20 may communicate sensor information to memory 19 via a wireless communication technique.

In some examples, sensor 20 may be positioned on distal portion 17A of elongated body 12 of medical device 10 including portions of elongated body 12 positioned distal to distal end 12A connected to a fluid collection container (e.g., a urine bag) or the like.

External device 24 may be a computing device, such as a workstation, a desktop computer, a laptop computer, a smart phone, a tablet, a server or any other type of computing device configured to receive, process and/or display sensor information including patient-specific information stored on memory 19. In some examples, external device 24 is a multi-parameter monitor configured to present information about other sensed parameters (e.g., blood oxygen saturation levels, blood pressure, heart rate, and the like). In other examples, external device 24 is a more specialized device, e.g., configured to present only the AKI information or a limited set of information including the Aki information. External device 24 may be configured to display information from sensor 20 (e.g., the amount of an AKI biomarker in urine within catheter 10 sensed by sensor 20 or an AKI status determined based on the amount of the AKI biomarker in urine sensed by sensor 20) to a user via a user interface display.

FIG. 2 is a diagram illustrating an example cross-section of catheter 10, where the cross-section is taken along line A-A in FIG. 1 in a direction orthogonal to central longitudinal axis 16. FIG. 2 depicts a cross section of elongated body 12, that defines lumen 34 and lumen 36. In some examples, lumen 34 may be referred to as a drainage lumen, such as in examples in which medical device 10 is a Foley catheter configured to drain urine from a bladder of a patient, and lumen 36 may be referred to as an inflation lumen in examples in which lumen 36 is configured to deliver an inflation fluid to anchoring member 18. Elongated body 12 may enclose connection 38.

Lumen 34 may serve as a passage for urine entering medical device 10 through fluid opening 13 to fluid opening 14A. Lumen wall 32 of elongated body 12 is positioned between lumen 36 and lumen 34, and outer wall 33 of elongated body 12, together with lumen wall 32 defines lumen 36. In some examples, elongated body 12 defining lumen walls 32, 33 are formed from the same material, such as silicone. In addition, as shown in FIG. 2, in some examples, lumen wall 32 extends around an entire outer perimeter of lumen 34 (e.g., an outer circumference in examples in that the inner perimeter is circular in cross-section).

Inflation lumen 36 may serve as a passage for a fluid, such as sterile water or saline, or a gas, such as air, from inflation opening 14B to anchoring mechanism 18. For example, an inflation device (not shown) may pump fluid or gas into inflation lumen 36 through inflation opening 14B into anchoring member 18 so anchoring member 18 is inflated to a size suitable to anchor medical device 10 to the patient's bladder. While inflation lumen 36 is shown as circular in cross section, it may be of any shape. In some examples, there may be a plurality of inflation lumens. For example, a plurality of inflation lumens may substantially surround lumen 34. In some examples, anchoring member 18 may be an expandable structure not an inflatable balloon. In such examples, inflation lumen 36 may be replaced by a deployment mechanism that may permit a clinician to expand the expandable structure. For example, inflation lumen may be replaced by a mechanical device pushed and pulled separately from the medical device 10 by a clinician to expand or retract the expandable structure.

Connection 38 may serve to connect sensor 20 positioned at distal portion 17A to memory 19 or more directly to processing circuitry of catheter 10 or external device 24. Connection 38 may be an electrical, optical, or other connection. In some examples, connection 38 may comprise a plurality of connections. For example, connection 38 may include one of more wired or optical connections to a temperature sensor and one or more connections to a pressure sensor. In some examples, connection 38 may include one or more power connections to power sensor 20 and one or more communications connections to receive sensor information from sensor 20.

As shown in FIG. 2, sensor 20 may be configured such that a surface of sensor 20 forms a surface of lumen 34 so that a fluid in lumen 34 may contact sensor 20. Such interaction may allow sensor 20 to determine an amount of AKI biomarker(s) in a fluid flowing in lumen 34. In the configuration of FIG. 2, sensor 20 may determine the level of AKI biomarker present in urine that flow through lumen 34 without the urine being removed from lumen 34 or elongate body 12 such that the urine that is sampled flow out of the distal end 12A via opening 14A into a collection bag. By analyzing the urine as it flow past sensor 20, sensor 20 may be able to monitor the amount of AKI biomarker on a substantially continuous and/or substantially real time basis (e.g., as compared to requiring a sample to be removed from the system and transported to a laboratory for analysis).

Information from sensor 20 regarding the sensed AKI biomarker levels may be stored in memory 19 and/or communicated to external device 24. External device 24 may then display the AKI biomarker information sensed by sensor 20 For example, external device 24 may display the concentration of one or more AKI biomarkers sensed by sensor 20 at one given point in time (e.g., as a single value) and/or over a period of time (e.g., as a plot of concentration versus time). As another example, external device 24 may display an AKI status determined by processing circuitry based on the concentration of one or more AKI biomarkers sensed by sensor 20. The AKI status can be, for example, an AKI status selected by the processing circuitry from a predetermined set of AKI statuses, each AKI status being associated with a particular AKI biomarker concentration or range of values.

A user such as a clinician may also control the operation of sensor 20 via external device 24. For example, a clinician may input an indication via external device 24 that sensor 20 should initiate, adjust, and/or suspend sensing of an AKI biomarker. Additionally, or alternatively, catheter device 10 may be configured to sense or otherwise determine a level of biomarker within the fluid using sensor 20 at preset time intervals (e.g., as scheduled by a clinician via external device) and/or based on a triggering event. For example, catheter device 10 may be configured to take samples (e.g., such as by processing circuitry controlling valves that control the introduction of fluid into a sampling lumen of catheter 10 to expose sensor 20 to the fluid and enable sensor 20 to sense a biomarker concentration in the sampled fluid) and determine the level of biomarker in a fluid within lumen 34 when urine output is determined to be below a defined threshold or when urine oxygen limits exceeded. Such determination may be made by other sensors on catheter device 10.

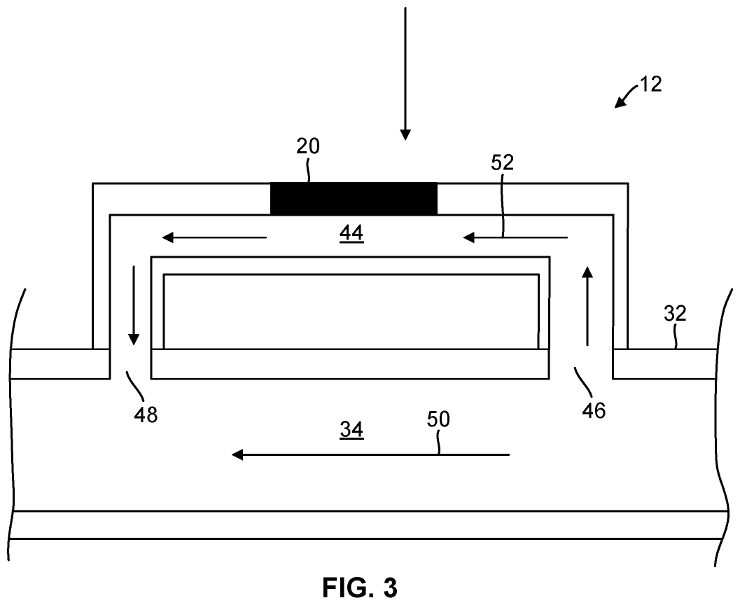

FIG. 3 is a schematic diagram illustrating another example of catheter device 10 including sensor 20 that is configured to sense an AKI biomarker in a fluid within elongate body 12. The example of FIG. 3 is a partial cross-sectional view along the longitudinal axis 16 of elongated body 12 (e.g., a cross-section that is substantially orthogonal to cross-section A-A of FIG. 1). For ease of illustration, the example of FIG. 3 is shown with a single wall 32 defining lumen 34 rather than also including lumen 36.

As described previously, a fluid such as urine may move along flow path 50 within lumen 34 of elongated body 12. Unlike the example of FIG. 2, elongated body 12 includes sampling lumen 44 that is fluidically couple to lumen 34. Lumen 34 may be referred to as the primary lumen and lumen 34 may extend substantially the entire length of elongate body 12. While only a single sampling lumen is shown in FIG. 2, in some examples, more than one sampling lumen may be included that is fluidically coupled to elongated body 12 (e.g., where each sampling lumen includes a separate AKI biomarker sensor configured to sense the same or a different AKI biomarker as sensor 20).

As shown in FIG. 3, sampling lumen 44 extends from an inlet port 46 to an outlet port 48, and is fluidically coupled to lumen 34. In some examples, sampling lumen 44 is defined by a tube that is coupled to elongated body 12 (e.g., either permanently or in a removable manner). In other examples, sampling lumen 44 is integrally formed with the rest of elongated body 12, e.g., via an injection molding process or other suitable process.

A portion of the fluid flowing within lumen 34 may be directed into sampling lumen 44 by entering sampling flow path 44 via inlet port 46. In the example of FIG. 3, the fluid in sampling lumen 44 flows along flow path 52 and rejoin flow path 50 in lumen 34 via outlet port 48. In this configuration, the fluid in sampling lumen 44 bypasses a portion of lumen 34 but still ultimately flows out of distal end 12A out outlet 14A (e.g., into a collection bag). In other examples, the fluid that enters sampling lumen 44 via inlet port 46 drains into another collection bag separate from the collection bag that lumen 34 drains into. In some examples, inlet port 46 and/or outlet port 48 includes a valve that may be selectively open and closed to control the flow of urine or other fluid into sampling lumen 44. The valve(s) may be manually controlled by a user and/or by processing circuity of catheter 10 and/or external device 24. The fluid within sampling lumen 44 may come into contact with sensor 20 (or otherwise interact with sensor 20) such that sensor 20 may sense the amount of one or more biomarkers within the fluid.

While the examples of FIGS. 2 and 3 include only a single sensor 20, in other examples, catheter 10 may include multiple sensors, e.g., with each individual sensor configured to sense the same or different AKI biomarkers. The multiple sensors can be longitudinally separated from each other (along longitudinal axis 16) and/or can be longitudinally aligned and distributed around an outer perimeter of elongated body 12 (e.g., circumferentially distributed in examples in which elongated body 12 is circular in cross-section).

Sensor 20 is configured to sense one or more AKI biomarkers in a fluid within lumen 34 using any suitable technique. In some examples, sensor 20 includes a competitive assay. Some example ways to build competitive assays use two half parts of the assay: an analyte binder and an analyte analogue called the ligand. These two compounds will, when having the correct concentrations, bind and create a binder-ligand complex. This complex may be separated by the analyte in question if the concentration of the analyte is high enough. Hence the overall sensor configuration utilizes measuring the amount and change in amount of the binder-ligand complex since this may only depend on the concentration of the analytes in question if the binder and ligand concentrations are kept constant, e.g., within a predefined and fixed volume of space defining a predefined volume of fluid.

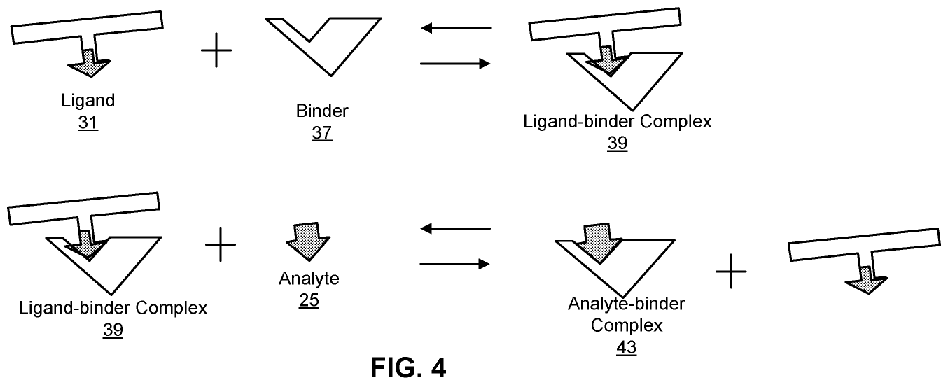

FIG. 4 is a conceptual diagram illustrating an example competitive assay that may be used by sensor 20 to sense an amount of AKI biomarker in urine of a patient flowing through lumen 34. A sample is shown in a predefined and fixed volume of space, e.g., sampling lumen 44 shown in FIG. 3. As shown, ligand 31 may bind to binder 37 forming a ligand-binder complex 39. This ligand-binder complex 39 can be separated by the analyte 25 creating a binder-analyte complex 43 and frees the ligand. The ability to measure the amount and change of amounts of the ligand-binder complex allow sensor 20 to determine the analyte concentration. Several methods to determine ligand-binder complex may also be employed. In some examples, sensor 20 outputs a signal that changes as a function of the amount of the ligand-binder complex in the predefined volume of space/predefined volume of fluid.

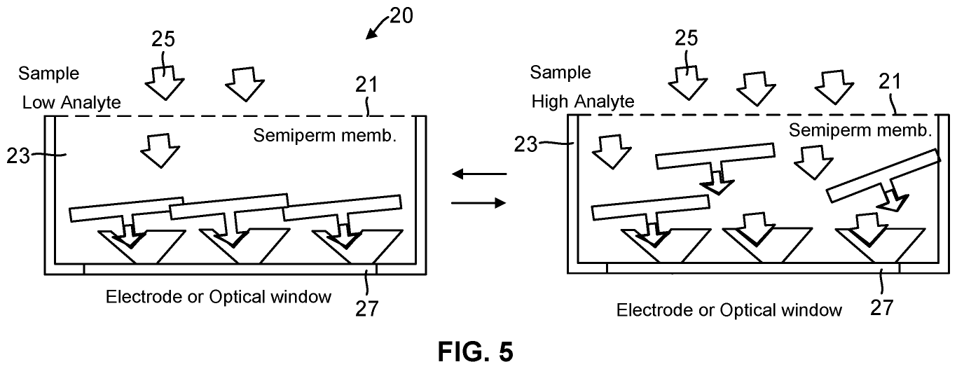

FIG. 5 is conceptual schematic diagram illustrating an example configuration of sensor 20 when using surface bound sensing principles for relatively low analyte concentration and comparatively high analyte concentration. As shown, sensor 20 includes a semipermeable membrane 21 that is configured to enable all or a portion of analyte 25 within a sample (e.g., urine within lumen 34 or lumen 44 (FIG. 3) that contacts sensor 20) to interact with the assay 23. Sensor 20 includes an electrode or optical window 27 that allows for sensor 20 to electrically or optically determine an amount of analyte 25 (e.g., an absolute or relative concentration) in the urine sample at a given point in time. Electrical changes from (competitive) binding assays can origin from changes in (complex) impedance when ligands are bound or displaced away from the electrode surface bound binder. Since the example assay is surface bound, the changes in impedance may origin from changes in what is sitting on the surface of the electrode, thus allowing for an amount of biomarkers in the fluid interacting with assay 23.

Using such an example, sensor 20 may monitor an amount of analyte 25 over a period of time. For example, one or more electrical characteristics (e.g., impedance) of the sample in the predefined volume of space may change as a function of the binder-ligand complex in the predefined volume of space, and sensor 20 can be configured to sense the one or more electrical characteristics of the sample. As another example, one or more optical characteristics (e.g., ability to absorb certain wavelengths of light, transparency, or the like) may change as a function of the binder-ligand complex in the predefined volume of space, and sensor 20 can be configured to sense the one or more optical characteristics of the sample.

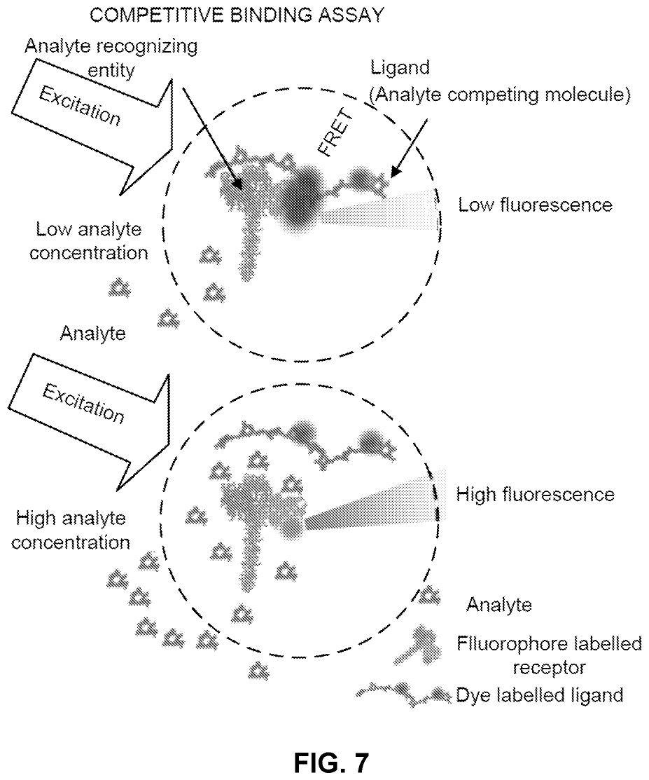

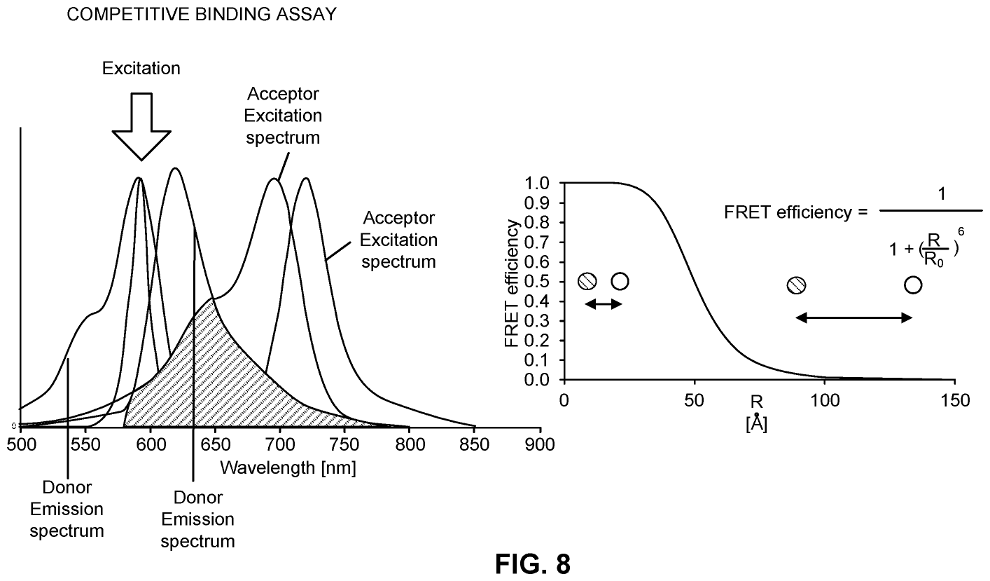

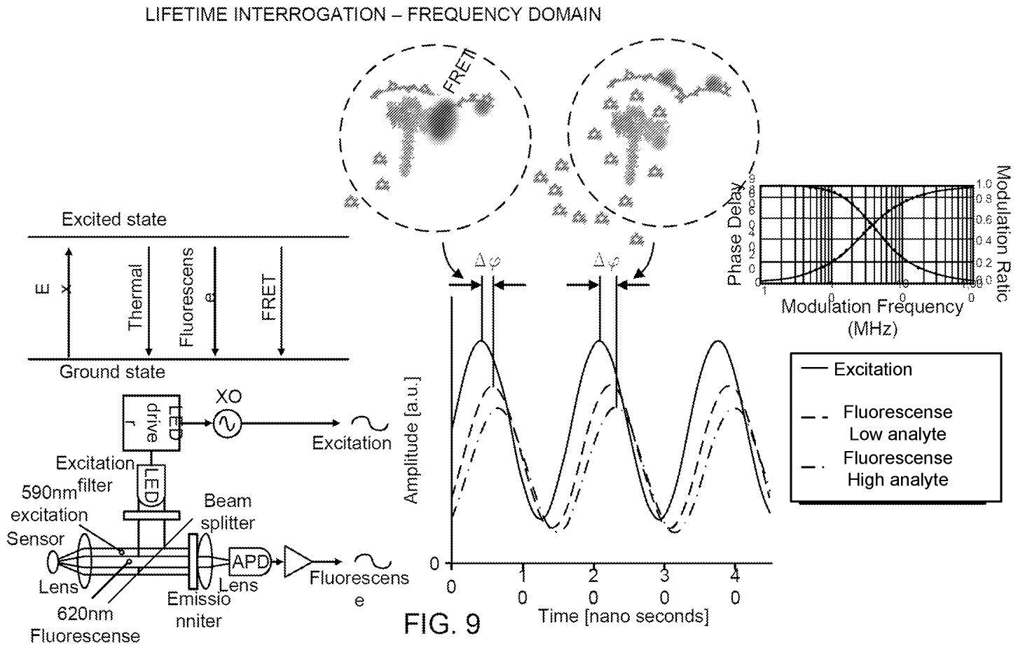

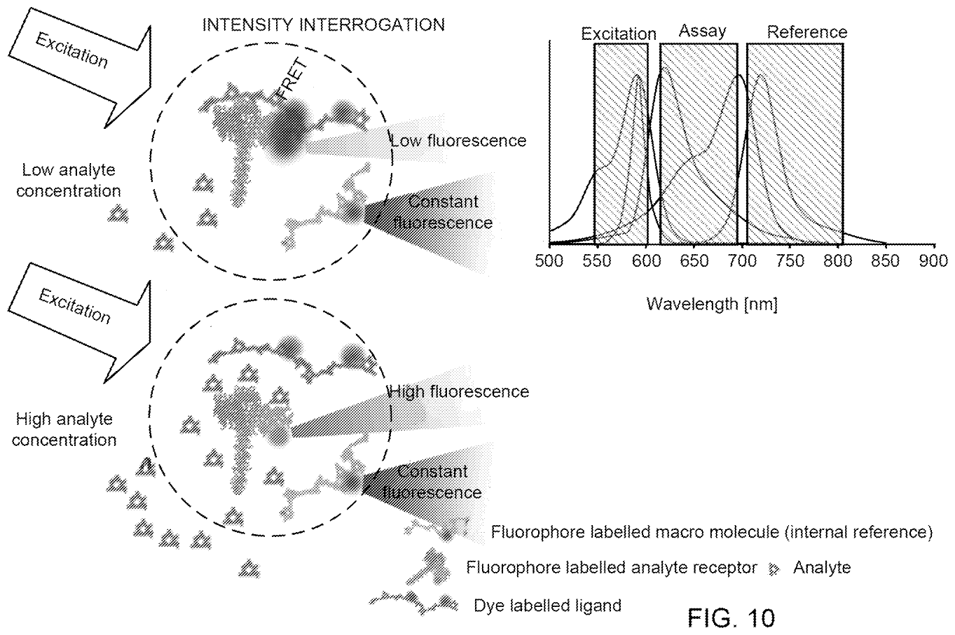

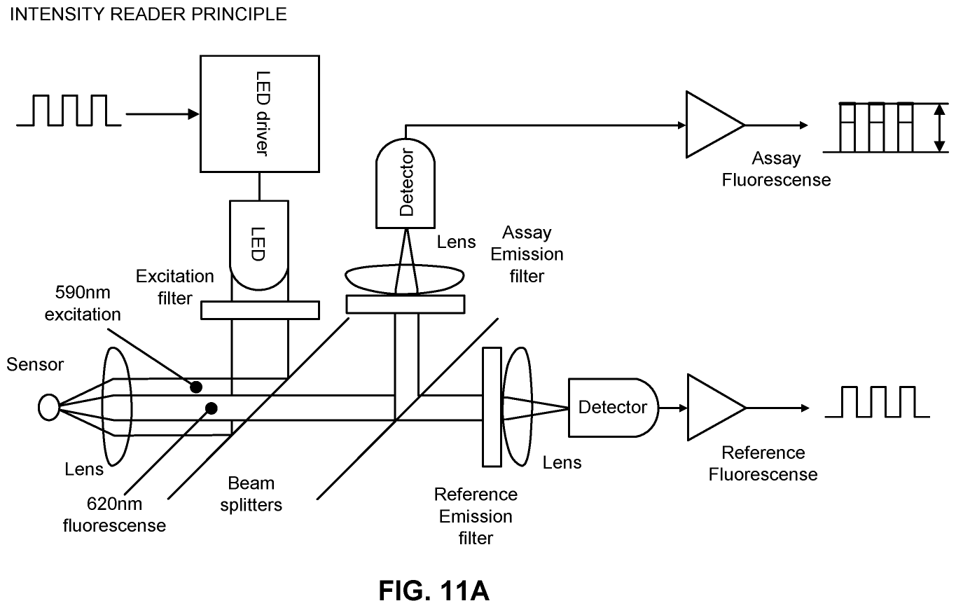

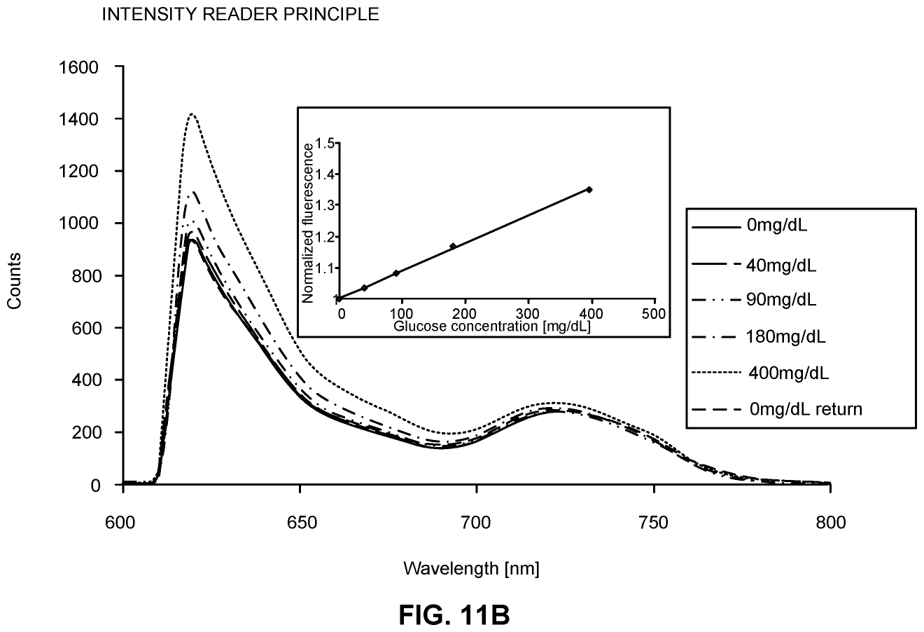

In another example, for an optical interrogation, the competitive assay may be labelled with a FRET pair (Förster Resonance Energy Transfer), including, consisting of or consisting essentially of a donor fluorophore and an acceptor fluorophore or dye (e.g., as shown in FIG. 7). The donor fluorophore is labelled onto either the receptor or the ligand and the acceptor fluorophore or dye is labelled onto the other assay component. The assay is excited in the excitation spectrum, which causes fluorescence emission from the donor fluorophore. The equilibrium state (e.g., a ratio between how many receptors are bound to the analyte versus how many are bound to the ligand) of the assay may be determined from the fluorescence emission or decay time, which is affected by FRET and which occurs when the donor fluorophore and the acceptor fluorophore/dye labelled onto the receptor and ligand are in close proximity (e.g., as indicated in FIG. 8, right side). The interrogation method may be any one or more of: 1) Frequency Domain Lifetime Interrogation, where the assay is excited using modulated light and the phase shift of the emitted relative to the excitation light is indicative of the decay time of the donor fluorophore (e.g., as shown in FIG. 9, right side). A schematic example of instrumentation is shown, e.g., in FIG. 9, right side; 2) Intensity interrogation (e.g., as shown in FIG. 10), which may include a reference fluorophore incorporated in the assay to help eliminate variations in the coupling between the assay and the optical instrumentation. An example of the instrumentation and circuitry that can be used is shown in FIG. 11A; and FIG. 3) Time Correlated Single Photon counting. FIG. 11B is a diagram illustrating example fluorescence emission sensed by the intensity interrogation instrumentation of FIG. 11A. For example, the competitive assay may be contained in a compartment by a membrane with a molecular weight cut-off, which allows for the analyte to pass the membrane, but not the assay components or bound to a hydrogel. In some examples, this assay may also be located at the distal end of an optical fiber and interrogated from the proximal end of the fiber. In some examples, a lifetime measurement—single photon counting technique may require spacing between photons; a few hundred thousand photons per time slot; and as low as, e.g., to a few seconds per measurement.

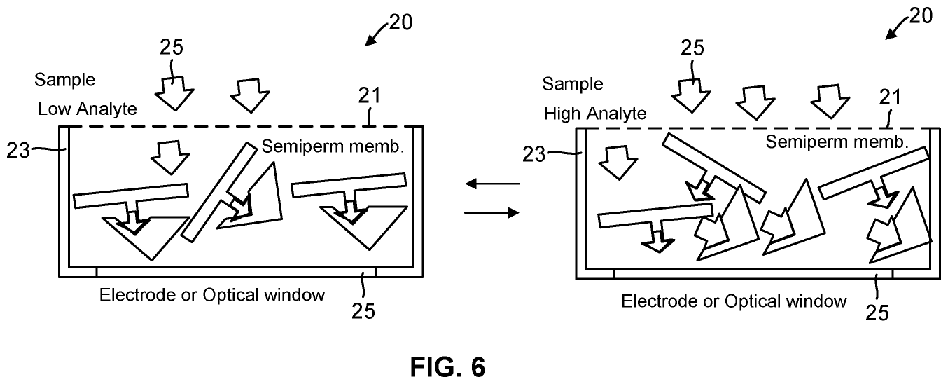

FIG. 6 is conceptual schematic diagram illustrating another example configuration of sensor 20. The example of FIG. 6 may be similar to that of the example of FIG. 5 but sensor 20 uses a homogenous assay (not surface bound) for the example of FIG. 6.

As described above, in some examples, the AKI biomarker(s) sensed by sensor 20 may include the proteins NGAL, TIMP-2, and/or IGFBP7. Table 1 below shows examples of how a competitive assay may be built for each of the three proteins. The ligand design can vary a lot and multiple epitopes to modify the ligand can be used depending on the binder. Epitope density on the ligand can be used to tailor the binding “strength” (avidity) between the binder and the ligand. Enterochelin and carboxymycobactin (CMB) respectively may both bind to the ligand binding cavity of NGAL. The latter (CMBs) however, may form a better fit into the ligand binding pocket of NGAL in some instances.

TABLE 1

| Analyte | Binder | Ligand |
|---|---|---|
| NGAL | Mono-clonal | rhNGAL (monomeric NGAL |
| (Siderocalin) | Anti-NGAL | recombinant human) modified |
| | Antibody | macromolecule e.g. HSA or Dextran |
| | NGAL | Siderophores-modified HSA or |
| | | other macromolecules e.g. Dextran |
| TIMP-2 | Mono-clonal | TIMP-2 (or fractions of TIMP-2) |
| | Anti-TIMP-2 | modified macromolecule e.g. HSA |
| | Antibody | or Dextran |
| | TIMP- | Gelatin or Collagen modified |
| | 2(Gelatinase-A) | macromolecule e.g. HSA or Dextran |
| IGFBP7 | Mono-clonal | IGFBP7 (or fractions of IGFBP7) |
| | Anti-IGFBP7 | modified macromolecule e.g. HSA |
| | Antibody | or Dextran |
| | IGFBP7 | Insulin or Insulin Like Growth |
| | | Factor 1 modified macromolecule |
| | | e.g. HSA or Dextran |

Creainine, Creatine and urea are other AKI biomarkers that may be sensed by sensor 20. Example technique for measuring smaller analytes like creatinine and urea using catheter 10 are described below.

In some examples, sensor 20 may be an amperometric sensor measuring creatinine using the three-enzyme conversion described below.

Reaction one catalyzed by Creatininase converting creatinine to Creatine:

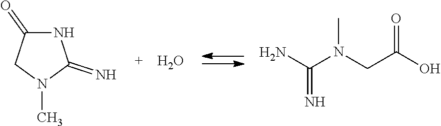

Reaction two catalyzed by Creatinase converting creatine to sacrosine:

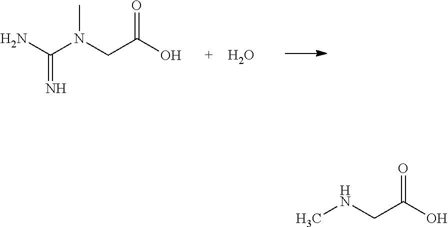

Third reaction catalyzed by Sacrosine Oxidase oxidizing sacrosine producing hydrogen peroxide:

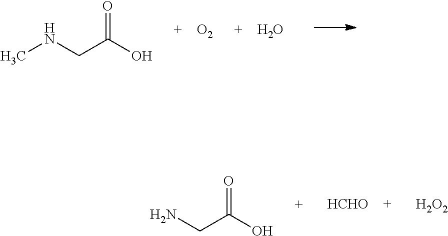

Hydrogen peroxide may be detected amperometrically by sensor 20 at a polarized anode (e.g., +700 mV vs. Ag/AgCl reference). The current may be proportional to the creatinine concentration, thus allowing sensor 20 to determine a concentration of creatine in urine within lumen 34.

In some examples, interference from creatine on the creatinine measurement may be, e.g., approximately 5%, hence one does not need to measure the creatine level in the urine sample. Since the measurement is done on the same sample (=patient) there will be no large and only slow changes in interference levels hence this type of interference should be of lesser concern to this type of device.

In some examples, a potentiometric creatinine determination may be performed by sensor 20. For a potentiometric creatinine determination, e.g., the enzyme Creatinine Deaminase will convert creatinine to N-methylhydantoin and ammonium as shown:

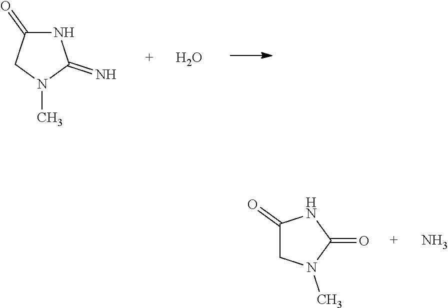

The ammonia in water will be present as ammonium depending on pH. The ammonium ion can be detected using an ammonium ion selective electrode (ISE). This setup may include a reference electrode in order to determine the electrical potential changes induced by the production of ammonium from the enzymatic conversion.

Most ammonium ISE's will exhibit potassium ion interference since the ion radii of potassium and ammonium are similar. Also, the amount of ammonium present in aqueous solution is highly dependent of pH. Using this potentiometric method of determining creatinine will call for the presence of a sample independent reference electrode (often build using a liquid junction), a potassium and a pH sensor in the same system and close to the creatinine sensor.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some respects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques may be fully implemented in one or more circuits or logic elements.

Various examples have been described. These and other examples are within the scope of the following clauses and claims.

Clause 1. A catheter comprising: an elongated body defining a lumen, the elongated body comprising a proximal portion and a distal portion; an anchoring member positioned on the proximal portion of the elongated body, the anchoring member configured to anchor the proximal portion of the elongated body to a patient; and a sensor configured to sense a biomarker in a fluid within the lumen of the elongated body, wherein the biomarker is indicative of an acute kidney infection (AKI) of the patient.

Clause 2. The catheter of clause 1, wherein the biomarker includes at least one of neutrophil gelatinase-associated lipocalin (NGAL), tissue inhibitor of metalloproteinase 2 (TIMP-2), or insulin-like growth factor binding protein 7 (IGFMP7).

Clause 3. The catheter of clauses 1 or 2, wherein the sensor is configured such that a surface of the sensor contacts the fluid within the lumen of the elongated body.

Clause 4. The catheter of any one of clauses 1 to 3, wherein the lumen includes a primary lumen and a sampling lumen fluidically coupled to the primary lumen.

Clause 5. The catheter of clause 4, wherein the sampling lumen bypasses a portion of the primary lumen.

Clause 6. The catheter of clause 4, further comprising at least one valve configured to the actuated to selectively allow the fluid from the primary lumen to flow into the sampling lumen.

Clause 7. The catheter of clause 4, wherein the sensor is positioned to interact with the fluid within the sampling lumen.

Clause 8. The catheter of any one of clauses 1 to 7, wherein the sensor comprises a first sensor and the biomarker comprises a first biomarker, the catheter further comprising a second sensor positioned on the elongated body, the second sensor configured to sense a second biomarker in the fluid within the lumen of the elongated body, wherein the second biomarker is indicative of acute kidney infection (AKI) of the patient.

Clause 9. The catheter of any one of clauses 1 to 8, wherein the sensor includes a competitive assay.

Clause 10. The catheter of clause 9, wherein the competitive assay comprises at least one of a homogenous assay or a surface bond assay.

Clause 11. The catheter of clause 9, wherein the sensor includes a semipermeable membrane configured to contact the fluid within the lumen, wherein the semipermeable membrane is permeable to the sensed biomarker.

Clause 12. The catheter of any one of clauses 1 to 11, wherein the sensor is configured to sense a concentration of a biomarker in a fluid within the lumen of the elongated body, and wherein the concentration of the biomarker is indicative of acute kidney infection (AKI) of the patient.

Clause 13. The catheter of clause 12, further comprising an external display configured to display an indication of the sensed concentration of the biomarker in the fluid or configured to display an indication of an AKI status of the patient determined based on the concentration of the biomarker.

Clause 14. The catheter of any one of clauses 1 to 13, wherein the sensor is positioned directly on the elongated body.

Clause 15. The catheter of any one of clauses 1 to 14, wherein the sensor comprises a first sensor, the catheter further comprising a second sensor configured to sense at least one of a temperature of the fluid, amount of dissolved oxygen of the fluid, flow rate of the fluid, or pressure of the fluid within the lumen.

Clause 16. The system of clause 15, wherein the first sensor is configured sense the biomarker in the fluid within the lumen of the elongated body based on the sensed at least one of the temperature of the fluid, the amount of dissolved oxygen of the fluid, the flow rate of the fluid, or the pressure of the fluid within the lumen.

Clause 17. A method comprising sensing, using the catheter of any one of clauses 1 to 16, the biomarker in the fluid within a lumen of a catheter.

Clause 18. A method comprising sensing, via a sensor, a biomarker in a fluid within a lumen of a catheter, the catheter comprising: an elongated body defining the lumen, the elongated body comprising a proximal portion and a distal portion; an anchoring member positioned on the proximal portion of the elongated body, the anchoring member configured to anchor the proximal portion of the elongated body to a patient; and the sensor configured to sense the biomarker in a fluid within the lumen of the elongated body, wherein the biomarker is indicative of an acute kidney infection (AKI) of the patient.

Clause 19. The method of clause 18, wherein the biomarker includes at least one of neutrophil gelatinase-associated lipocalin (NGAL), tissue inhibitor of metalloproteinase 2 (TIMP-2), or insulin-like growth factor binding protein 7 (IGFMP7).

Clause 20. The method of clauses 18 or 19, wherein the sensor is configured such that a surface of the sensor contacts the fluid within the lumen of the elongated body.

Clause 21. The method of any one of clauses 1 to 3, wherein the lumen includes a primary lumen and a sampling lumen fluidically coupled to the primary lumen.

Clause 22. The method of clause 21, wherein the sampling lumen bypasses a portion of the primary lumen.

Clause 23. The method of clause 21, wherein the catheter comprises at least one valve, wherein sensing the biomarker in the fluid comprises actuating the at least one valve to selectively allow the fluid from the primary lumen to flow into the sampling lumen.

Clause 24. The method of clause 21, wherein the sensor is positioned to interact with the fluid within the sampling lumen.

Clause 25. The method of any one of clauses 18 to 24, wherein the sensor comprises a first sensor and the biomarker comprises a first biomarker, the catheter comprising a second sensor positioned on the elongated body, the method further comprising sensing, via the second sensor, a second biomarker in the fluid within the lumen of the elongated body, wherein the second biomarker is indicative of acute kidney infection (AKI) of the patient.

Clause 26. The method of any one of clauses 18 to 25, wherein the sensor includes a competitive assay.

Clause 27. The method of clause 26, wherein the competitive assay comprises at least one of a homogenous assay or a surface bond assay.

Clause 28. The method of clause 26, wherein the sensor includes a semipermeable membrane configured to contact the fluid within the lumen, wherein the semipermeable membrane is permeable to the sensed biomarker.

Clause 29. The method of any one of clauses 18 to 28, wherein sensing the biomarker in the fluid comprises sensing a concentration of a biomarker in the fluid within the lumen of the elongated body, and wherein the concentration of the biomarker is indicative of acute kidney infection (AKI) of the patient.

Clause 30. The method of clause 29, wherein the catheter comprises an external display configured to display an indication of the sensed concentration of the biomarker in the fluid or configured to display an indication of an AKI status of the patient determined based on the concentration of the biomarker.

Clause 31. The method of any one of clauses 18 to 30, wherein the sensor is positioned directly on the elongated body.

Clause 32. The method of any one of clauses 18 to 31, wherein the sensor comprises a first sensor, the catheter further comprising a second sensor, the method further comprising sensing, via the second sensor, at least one of a temperature of the fluid, amount of dissolved oxygen of the fluid, flow rate of the fluid, or pressure of the fluid within the lumen.

Clause 33. The system of clause 32, wherein sensing the biomarker in the fluid comprises sensing the biomarker in the fluid within the lumen of the elongated body based on the sensed at least one of the temperature of the fluid, the amount of dissolved oxygen of the fluid, the flow rate of the fluid, or the pressure of the fluid within the lumen.

1. A catheter comprising:
an elongated body defining a lumen, the elongated body comprising a proximal portion and a distal portion, wherein the lumen includes a primary lumen and a sampling lumen fluidically coupled to the primary lumen through an inlet port and an outlet port positioned distally to the inlet port;
an anchoring member positioned on the proximal portion of the elongated body, the anchoring member configured to anchor the proximal portion of the elongated body to a patient; and
a sensor configured to sense a biomarker in a fluid within the lumen of the elongated body, wherein the biomarker is indicative of an acute kidney infection (AKI) of the patient.

2. The catheter of claim 1, wherein the biomarker includes at least one of neutrophil gelatinase-associated lipocalin (NGAL), tissue inhibitor of metalloproteinase 2 (TIMP-2), or insulin-like growth factor binding protein 7 (IGFMP7).

3. The catheter of claim 1, wherein the sensor is configured such that a surface of the sensor contacts the fluid within the lumen of the elongated body.

4. The catheter of claim 1, wherein the sampling lumen bypasses a portion of the primary lumen.

5. The catheter of claim 1, further comprising at least one valve configured to be actuated to selectively allow the fluid from the primary lumen to flow into the sampling lumen through the inlet port.

6. The catheter of claim 1, wherein the sensor is positioned to interact with the fluid within the sampling lumen.

7. The catheter of claim 1, wherein the sensor comprises a first sensor and the biomarker comprises a first biomarker, the catheter further comprising a second sensor positioned on the elongated body, the second sensor configured to sense a second biomarker in the fluid within the lumen of the elongated body, wherein the second biomarker is indicative of acute kidney infection (AKI) of the patient.

8. The catheter of claim 1, wherein the sensor includes a competitive assay.

9. The catheter of claim 8, wherein the competitive assay comprises at least one of a homogenous assay or a surface bond assay.

10. The catheter of claim 8, wherein the sensor includes a semipermeable membrane configured to contact the fluid within the lumen, wherein the semipermeable membrane is permeable to the sensed biomarker.

11. The catheter of claim 1, wherein the sensor is configured to sense a concentration of a biomarker in a fluid within the lumen of the elongated body, and wherein the concentration of the biomarker is indicative of acute kidney infection (AKI) of the patient.

12. The catheter of claim 11, further comprising an external display configured to display an indication of the sensed concentration of the biomarker in the fluid or configured to display an indication of an AKI status of the patient determined based on the concentration of the biomarker.

13. The catheter of claim 1, wherein the sensor comprises a first sensor, the catheter further comprising a second sensor configured to sense at least one of a temperature of the fluid, amount of dissolved oxygen of the fluid, flow rate of the fluid, or pressure of the fluid within the lumen.

14. The catheter of claim 13, wherein the first sensor is configured sense the biomarker in the fluid within the lumen of the elongated body based on the sensed at least one of the temperature of the fluid, the amount of dissolved oxygen of the fluid, the flow rate of the fluid, or the pressure of the fluid within the lumen.

15. A method of sensing a biomarker, the method comprising:
sensing a biomarker indicative of an acute kidney infection (AKI) of a patient in a fluid within a lumen of an elongated body defining the lumen, the elongated body comprising a proximal portion and a distal portion, wherein the lumen includes a primary lumen and a sampling lumen fluidically coupled to the primary lumen through an inlet port and an outlet port positioned distally to the inlet port, and wherein an anchoring member is positioned on the proximal portion of the elongated body, the anchoring member configured to anchor the proximal portion of the elongated body to a patient.

16. The method of claim 15, wherein the biomarker includes at least one of neutrophil gelatinase-associated lipocalin (NGAL), tissue inhibitor of metalloproteinase 2 (TIMP-2), or insulin-like growth factor binding protein 7 (IGFMP7).

17. The method of claim 15, wherein sensing the biomarker comprises contacting, by a surface of the sensor, the fluid within the lumen of the elongated body.

18. The method of claim 15, wherein the sampling lumen bypasses a portion of the primary lumen.

19. The method of claim 15, further comprising actuating at least one valve to selectively allow the fluid from the primary lumen to flow into the sampling lumen through the inlet port.

20. The method of claim 15, wherein sensing the biomarker comprises interacting with the fluid within the sampling lumen.
